(19) United States
(12) Patent Application Publication
(10) Pub. No.: US 2014/0222034 A1
(43) Pub. Date: Aug. 7, 2014

(54) SUTURE PASSERS

(71) Applicant: Justin D. SALIMAN, Los Angeles, CA (US)

(72) Inventor: Justin D. SALIMAN, Los Angeles, CA (US)

(21) Appl. No.: 13/759,006

(22) Filed: Feb. 4, 2013

(51) Int. Cl. A61B 17/04

(52) U.S. Cl. CPC A61B 17/0469
USPC 606/145

(57) ABSTRACT

Suture passer devices, including suture passers configured with an axially slideable jaw that includes a tissue-penetrating distal end region. Also described are suture passers including jaws housing tissue penetrating needles to pass suture that are substantially thin. Methods of using such devices to pass a suture through tissue are provided.

### CROSS REFERENCE TO RELATED APPLICATIONS

This application does not claim priority to any other patent application.

The suture passers and methods of suturing described herein may related to, and may incorporate any of the features or elements described in the following patent applications, each of which is herein incorporated by reference in its entirety. Specifically: U.S. patent application Ser. No. 11/773,388, filed on Jul. 3, 2007, titled “METHODS AND DEVICES FOR CONTINUOUS SUTURE PASSING,” now Publication No. US-2009-0012538-A1; U.S. patent application Ser. No. 12/972,222, filed on Dec. 17, 2010, titled “METHODS AND DEVICES FOR CONTINUOUS SUTURE PASSING,” now Publication No. US-2011-0087246-A1; U.S. patent application Ser. No. 13/462,760, filed on May 2, 2012, titled “METHODS OF MENISCUS REPAIR,” now Publication No. US-2012-0239062-A1; U.S. patent application Ser. No. 13/006,966, filed on Jan. 14, 2011, titled “METHODS FOR CONTINUOUS SUTURE PASSING,” now Publication No. US-2011-0130773-A1; U.S. patent application Ser. No. 13/090,089, filed on Apr. 19, 2011, titled “METHODS OF MENISCUS REPAIR,” now Publication No. US-2011-0218557-A1; U.S. patent application Ser. No. 12/291,159, filed on Nov. 5, 2008, titled “SUTURE PASSING INSTRUMENT AND METHOD,” now Publication No. US-2010-0331863-A2; U.S. patent application Ser. No. 12/972,168, filed on Dec. 17, 2010, titled “SUTURE PASSING INSTRUMENT AND METHOD,” now Publication No. US-2011-0152892-A1; U.S. patent application Ser. No. 13/062,664, filed on Apr. 19, 2011, titled “KNOTLESS SUTURE ANCHORS,” now Publication No. US-2011-0190815-A1; U.S. patent application Ser. No. 12/620,029, filed on Nov. 17, 2009, titled “METHODS OF SUTURING AND REPAIRING TISSUE USING A CONTINUOUS SUTURE PASSER DEVICE,” now Publication No. US-2010-0130990-A1; U.S. patent application Ser. No. 12/942,803, filed on Nov. 9, 2010, titled “DEVICES, SYSTEMS AND METHODS FOR MENISCUS REPAIR,” now Publication No. US-2011-0112556-A1; U.S. patent application Ser. No. 13/462,728, filed on May 2, 2012, titled “DEVICES, SYSTEMS AND METHODS FOR MENISCUS REPAIR,” now Publication No. US-2012-0265221-A1; U.S. patent application Ser. No. 13/114,983, filed on May 24, 2011, titled “SUTURING AND REPAIRING TISSUE USING IN VIVO SUTURE LOADING,” now Publication No. US-2011-0270280-A1; U.S. patent application Ser. No. 13/347,184, filed on Jan. 10, 2012, titled “IMPLANT AND METHOD FOR REPAIR OF THE ANTERIOR CRUCIATE LIGAMENT,” now Publication No. US-2012-0179254-A1; U.S. patent application Ser. No. 13/247,892, filed on Sep. 28, 2011, titled “MENISCUS REPAIR,” now Publication No. US-2012-0283750-A1; U.S. patent application Ser. No. 13/323,391, filed on Dec. 12, 2011, titled “SUTURE PASSER DEVICES AND METHODS,” now Publication No. US-2012-0283753-A1; and U.S. patent application Ser. No. 13/462,773, filed on May 2, 2012, titled “SUTURE PASSER DEVICES AND METHODS,” now Publication No. US-2012-0283754-A1, each of which is incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present invention relates to suture passers, suturing techniques, devices and methods, for surgical use and methods of repairing tissue. More particularly, described herein are suture passers that may be used for performing arthroscopic (including minimally invasive, e.g., endoscopic) procedures.

### BACKGROUND

Suturing of tissue during surgical procedures is time consuming and can be particularly challenging in difficult to access body regions and regions that have limited clearance, such as regions partially surrounded or covered by bone. For many surgical procedures, it is necessary to make a large opening in the human body to expose the area requiring surgical repair. However, in many cases, accessing the tissue in this manner is undesirable, increasing recovery time, and exposing the patient to greater risk of infection.

Suturing instruments (“suture passers” or “suturing devices”) have been developed to assist in accessing and treating internal body regions, and to generally assist a physician in repairing tissue. Although many such devices are available for endoscopic and/or percutaneous use, these devices suffer from a variety of problems, including limited ability to navigate and be operated within the tight confines of the body, risk of injury to adjacent structures, problems controlling the position and/or condition of the tissue before, during, and after passing the suture, as well as problems with the reliable functioning of the suture passer.

For example, some surgical instruments used in endoscopic procedures are limited by the manner in which they access the areas of the human body in need of repair. In particular, the instruments may not be able to access tissue or organs located deep within the body or that are in some way obstructed. In addition, many of the instruments are limited by the way they grasp tissue, apply a suture, or recapture the needle and suture. Furthermore, many of the instruments are complicated and expensive to use due to the numerous parts and/or subassemblies required to make them function properly. Suturing remains a delicate and time-consuming aspect of most surgeries, including those performed endoscopically.

For example, some variations of suture passers, such as those described in U.S. Pat. No. 7,377,926 to Taylor, have opposing jaws that open and close over tissue. One, or in some variations, both, jaws open, scissor-like, so that tissue may be inserted between the open jaws. Unfortunately, such devices cannot be adequately positioned for use in hard to navigate body regions such as the joints of the body, including the knee (e.g., meniscus) and the shoulder.

The meniscus is a C-shaped piece of fibrocartilage which is located at the peripheral aspect of the joint (e.g., the knee) between the condyles of the femur and the tibia on the lateral and medial sides of the knee. The central two-thirds of the meniscus has a limited blood supply while the peripheral one third typically has an excellent blood supply. Acute traumatic events commonly cause meniscus tears in younger patients while degenerative tears are more common in older patients as the menisci become increasingly brittle with age. Typically, when the meniscus is damaged, a torn piece of meniscus may move in an abnormal fashion inside the joint, which may lead to pain and loss of function of the joint. Early arthritis can also occur due to these tears as abnormal mechanical movement of torn meniscal tissue and the loss of the shock absorbing properties of the meniscus lead to destruction of the surrounding articular cartilage. Occasionally, it is possible to repair a torn meniscus. While this may be done arthroscopically, surgical repair using a suture has proven difficult to perform because of the hard-to-reach nature of the region and the difficulty in placing sutures in a way that compresses and secures the torn surfaces.

Arthroscopy typically involves inserting a fiberoptic telescope that is about the size of a pencil into the joint through an incision that is approximately ⅛ inch long. Fluid may then be inserted into the joint to distend the joint and to allow for visualization of the structures within that joint. Then, using miniature instruments which may be as small as 1/10 of an inch, the structures are examined and the surgery is performed.

The meniscus of the knee is just one example of a tissue that is difficult to access so that appropriate suturing may be performed. FIGS. 1A, 1B and 2 illustrate the anatomy of the meniscus in the context of a knee joint. As shown in FIG. 2 the capsule region (the outer edge region of the meniscus) is vascularized. Blood enters the meniscus from the menisculocapsular region 211 lateral to the meniscus. A typical meniscus has a flattened bottom (inferior surface or side) and a concave top (superior surface or side), and the outer cross-sectional shape is somewhat triangular. The outer edge of the meniscus transitions into the capsule. FIG. 3 illustrates the various fibers forming a meniscus. As illustrated in FIG. 3, there are circumferential fibers extending along the curved length of the meniscus, as well as radial fibers, and more randomly distributed mesh network fibers. Because of the relative orientations and structures of these fibers, and the predominance of circumferential fibers, it may be beneficial to repair the meniscus by suturing radially (vertically) rather than longitudinally or horizontally, depending on the type of repair being performed.

For example, FIGS. 4A-4E illustrate various tear patterns or injuries to a meniscus. Tears may be vertical/longitudinal (FIG. 4A), oblique (FIG. 4B), degenerative (FIG. 4C), including radially degenerative, transverse or radial (FIG. 4D) and horizontal (FIG. 4E). Most prior art devices for suturing or repairing the meniscus are only capable of reliably repairing vertical/longitudinal tears. Such devices are not typically useful for repairing radial or horizontal tears. Furthermore, prior art device mechanisms have a high inherent risk for iatrogenic injury to surrounding neurovascular structures and chondral surfaces.

Thus, there is a need for methods, devices and systems for suturing tissue, particularly tissue in difficult to access regions of the body including the joints (shoulder, knee, etc.). In particularly, it has proven useful to provide a device that may simply and reliably reach and pass sutures within otherwise inaccessible tissue regions. Such devices should be extremely low profile. Finally, it is useful to provide suturing devices that allow selective and specific penetration of the tissue by both the tissue penetrator (needle element) and a jaw so that complex (including right-angled) suturing patterns may be achieved. The methods, devices and systems described herein may address this need.

### SUMMARY OF THE DISCLOSURE

The present invention relates to suture passers. In particular, described herein are suture passer devices having a bent or bendable first jaw extending from an elongate body, and a second jaw that is independently axially slideable relative to the elongate body (and/or first jaw) to form a distal-facing opening between the first and second jaws into which target tissue may be held and sutured by extending a tissue-penetrator (e.g., needle) between the first and second jaws.

The first or second jaw may hold the tissue penetrator within an internal passage, and the tissue penetrator may be extended between the distal-facing opening to push and/or pull a suture between the first and second jaws. The tissue penetrator may be any appropriate material, but shape memory materials (e.g., shape memory alloys, plastics, etc.) are of particularly interest. The tissue penetrator may have a sharp (e.g., pointed, beveled, etc.) distal tip for penetrating tissue. The tissue penetrator may be biased (e.g., pre-bent) in a curve or bend. In general the tissue penetrator (e.g., needle) may extend from a side region of the first or second jaw, extend across the distal-facing opening, and connect to an opening on the side region of the opposite (e.g., second or first) jaw from which it extends. This opening may include a suture capture region that holds the suture passed by the tissue penetrator. The suture capture region may be a suture retainer that holds the suture when passed by the tissue penetrator. For example, the suture retainer may be a deflecting or deflectable clamping region, a hook, or the like.

In general, the tissue penetrator may be configured to bend as it extends from the jaw and across the distal-facing opening. For example, the tissue penetrator may be pre-biased to assume a bent or curved configuration as it extends from within a jaw. Thus, the tissue penetrator may extend approximately perpendicular to the side of the jaw housing it. In some variations the jaw includes a tissue penetrator deflection (e.g., ramped) region that helps deflect the jaw. In some variations the jaw housing the tissue penetrator does not include a deflector.

In some variations described herein, one or the other jaws, and particularly the axially slideable jaw, has a tissue penetrating distal tip region. The tissue penetrating distal tip region may be sharp, including pointed, beveled, wedge-shaped, or otherwise configured to cut into and/or through the tissue as it is extended distally. For example, the diameter of the tissue penetrating distal tip region may be small, allowing it to cut into the tissue. In some variations the tissue-penetrating distal tip region may be knife-like and/or needle-like. In some variations the tissue-penetrating distal tip region may be configured to apply energy to cut or pierce the tissue. For example, the distal tip region may be configured to apply RF energy and/or thermal energy, and/or ultrasound energy, and/or the like to cut and/or ablate the tissue allowing the second jaw to penetrate into the tissue. In some variations the jaw may also be configured to reduce bleeding (e.g., by cauterizing the tissue as it is cut).

For example, described herein are suture passer devices for passing a suture comprising: an elongate body extending distally and proximally along a long axis; a first jaw extending from a distal end region of the elongate body wherein the first jaw is bent or bendable at an angle relative to the long axis; a second jaw having a sharp, tissue penetrating distal tip, wherein the second jaw is configured to slide axially along the long axis distally and proximally relative to the elongate body, further wherein the first jaw and the second jaw form a distal-facing opening when the second jaw is extended distally and wherein the second jaw is retractable proximally so that it does not form the distal-facing opening with the first jaw; and a tissue penetrator configured to extend across the distal-facing opening between the first jaw and the second jaw to pass a suture there between.

As mentioned, the tissue penetrating distal tip of the second jaw may include a sharp distal tip. For example, the tissue penetrating distal tip of the second jaw may comprise a sharp point at the distal tip, a sharp edge, etc. In some variations only one of the jaws has a sharp tip, while the other jaw has a non-tissue penetrating (e.g., atraumatic) tip. For example, the first jaw may comprise an atraumatic distal tip region.

In some variations the first jaw is bendable. For example, the first jaw may be hinged to the distal end region of the elongate body. In some variations, the first jaw is bent or bendable at a predetermined angle relative to the long axis of the elongate body (e.g., approximately 10°, 15°, 20°, 25°, 30°, 35°, 40°, 45°, 50°, 55°, 60°, 65°, 70°, 75°, 80°, 85°, 90°, 95°, etc.). In some variations the first jaw has a fixed bend relative to the long axis of the elongate body.

The second jaw may be configured to retract into the elongate body. In some variations, the second jaw does slides axially relative to the elongate body, but does not retract into/out of the elongate body. For example, the second jaw may slide along an outer surface of the elongate body and/or into a housing region adjacent to the elongate body. The second jaw may be configured to be retracted partially or completely relative to the distal end of the elongate body. For example, in some variations, the distal tip of the second jaw may be retracted completely into the distal tip region of the elongate body.

Any of the variations of the devices described herein may include a handle. For example, a device may include a handle at a proximal end region of the elongate body. The handle may include one or more controls for actuating the first jaw, second jaw, and tissue penetrator.

In general, any appropriate tissue penetrator may be used. As mentioned above, the tissue penetrator may be configured as a needle, a ribbon, or the like. For example, the tissue penetrator may be configured as a strip of shape-memory material. The tissue penetrator may include a suture holding (retaining) region. For example, the tissue penetrator may include a hook, eyelet, cavity, or the like the hold a suture as it is passed between the first and second jaws. As mentioned, a tissue penetrator may be housed in the second jaw and configured to extend from an opening on the side of the second jaw.

Also described herein are methods of suturing tissue using a suture passer that has a sharp and tissue penetrating jaw. The devices described above may be used to pass a suture in an L-shaped configuration (or other angled configuration) within a tissue. For example, any of the devices described herein with a tissue-penetrating jaw may be used to pass a suture in multiple directions within the tissue.

For example, a suture passer as described herein may be used in a method of suturing tissue. In some variations a method of suturing tissue using a suture passer having an elongate body, a first jaw bent or bendable relative to the elongate body, and a sharp, tissue penetrating axially slideable second jaw, may include some or all of the steps of: positioning the first jaw of the suture passer adjacent a tissue with the second jaw retracted proximally relative to the elongate body; axially sliding the second jaw distally to penetrate the tissue by extending the second jaw distally by relative to the elongate body; extending a tissue penetrator through the tissue between the first and second jaws to pass a suture between the first and second jaws; retracting the tissue penetrator from between the first and second jaws; and sliding the second jaw proximally out of the tissue while leaving the suture in the tissue.

In general, methods of suturing the tissue may include placing an atraumatic jaw adjacent the tissue, then penetrating the tissue with the sharp (tissue penetrating) jaw and then extending a tissue penetrator (e.g., needle) between the two jaws to pass a suture between the jaws. Retracting the suture passer may leave the suture passed through the tissue in the path taken by the tissue penetrator and tissue-penetrating jaw.

In some variations, positioning the first jaw may comprise bending the first jaw relative to a long axis of the elongate body. Positioning the first jaw may comprises positioning the first jaw of the suture passer with a sharp tissue penetrating distal tip of the second jaw retracted into the elongate body.

Axially sliding the second jaw may comprise extending a sharp, tissue penetrating distal tip region of the second jaw into the tissue. The step of extending the tissue penetrator through the tissue may comprise extending a tissue penetrator from a side of the second jaw to a side of the first jaw. In some variations, extending the tissue penetrator comprises extending the tissue penetrator in a curve between the first and second jaws. In general, extending the tissue penetrator comprises pushing a suture between the first and second jaws. The suture may be retained in the opposite (E.g., first jaw).

In some variations, retracting the tissue penetrator comprises retracting the tissue penetrator into the second jaw.

Any of the methods described herein may be performed minimally invasively, including arthroscopically. For example, the first jaw may be positioned arthroscopically, and each of the steps thereafter may be performed arthroscopically thereafter.

### BRIEF DESCRIPTION OF THE DRAWINGS

FIGS. 1A and 1B illustrate the anatomy of the meniscus, one exemplary tissue that may be sutured using the devices described herein.

FIG. 2 illustrates the anatomy of the meniscus, including the capsule and associated vascular tissue.

FIG. 3 illustrates the structure of a meniscus.

FIGS. 4A-4E illustrate various tear patterns that may be repaired using the invention described herein.

FIGS. 5A-5C show one variation of a suture passer.

FIGS. 6A, 6B, and 6D show top and two side perspective views, respectively of the distal end of the suture passer shown in FIG. 5A.

FIG. 6C illustrates the arrangement of the tissue penetrator and suture stripper in the distal end region of the suture passer of FIG. 5A.

FIGS. 7A-7C show a suture stripper including a stripper plate (FIG. 7B) and base (FIG. 7C).

FIGS. 8A and 8B show side perspective views of the distal end region of a jaw including a suture stripper.

FIGS. 9A-9C show variations of jaws (e.g., second jaws) of a suture passer. The variation shown in FIG. 9A includes a deflection region for deflecting a tissue penetrator at the widest point of the jaw profile. FIG. 9D shows the variation of FIG. 9A with the tissue penetrator extended. FIG. 9E shows the variation of FIG. 9B with the tissue penetrator extended. FIG. 9F shows the variation of FIG. 9C with the tissue penetrator extended.

FIG. 10A shows a variation of a jaw with an atraumatic (non-tissue penetrating) distal tip region. FIGS. 10B and 10C show variations of a jaw with a tissue-penetrating distal tip region.

FIGS. 11A to 11E illustrate the distal end of one variation of a suture passer having a tissue-penetrating (e.g., sharp) jaw.

FIGS. 12A-12G illustrate one method of using a suture passer having a tissue-penetrating distal tip on one of the jaws.

FIGS. 13A-13F illustrate another variation of a method of using a suture passer having a tissue-penetrating distal tip.

FIGS. 14A-14F illustrate another variation of a suture passer.

FIG. 14G shows the suture passer of FIGS. 14A-14F used to pass a suture through a meniscus of a knee.

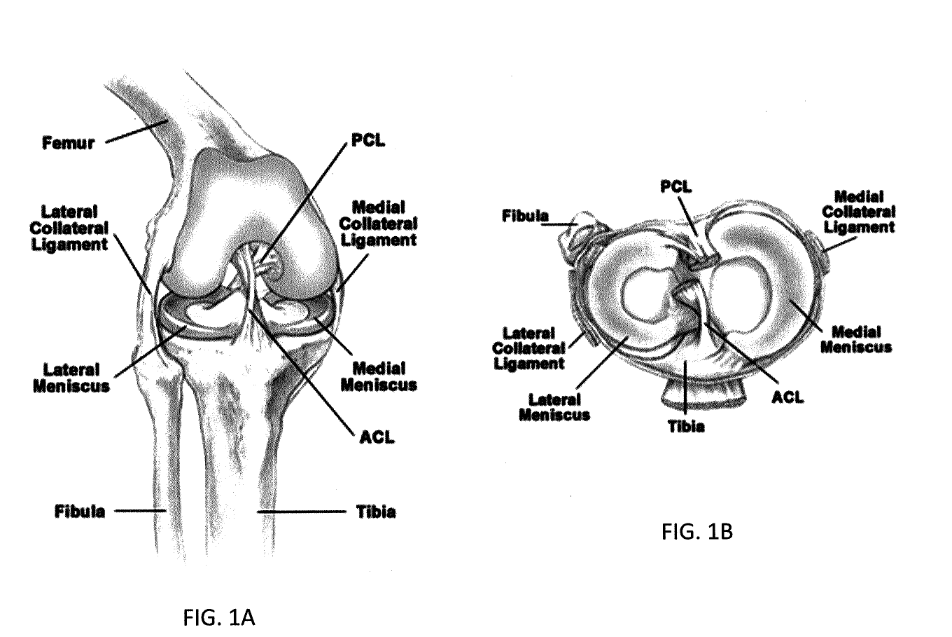

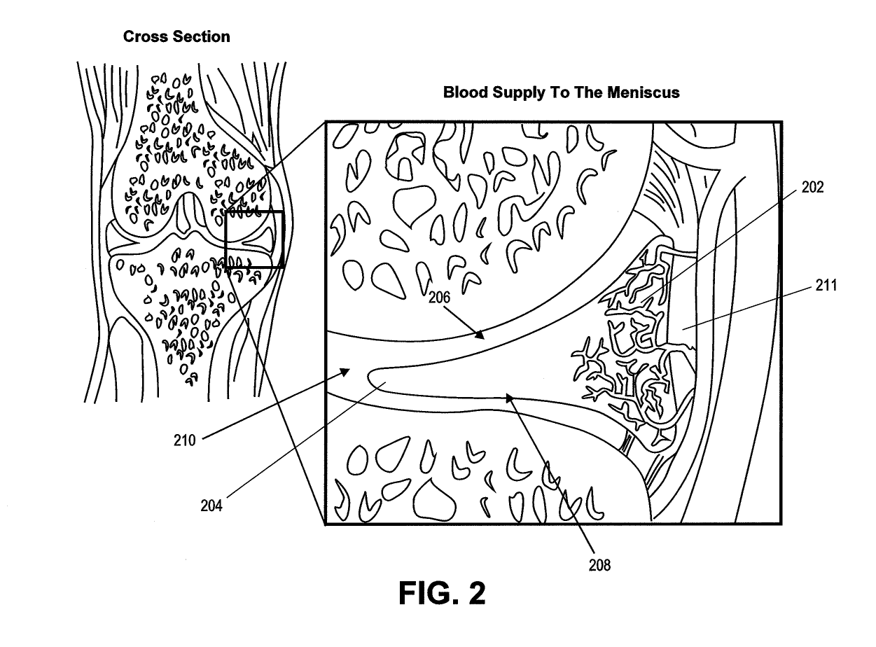

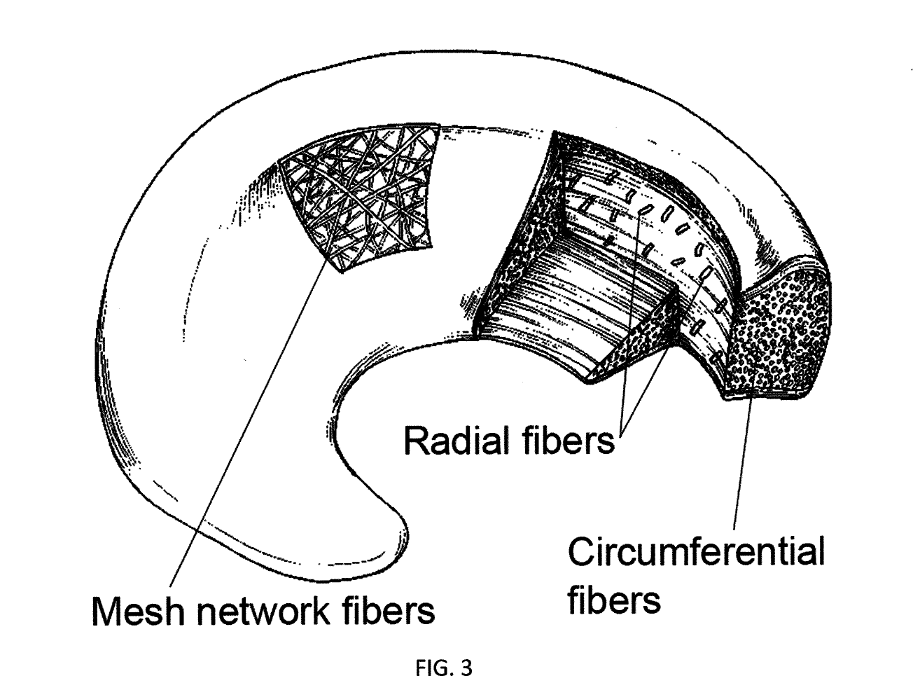

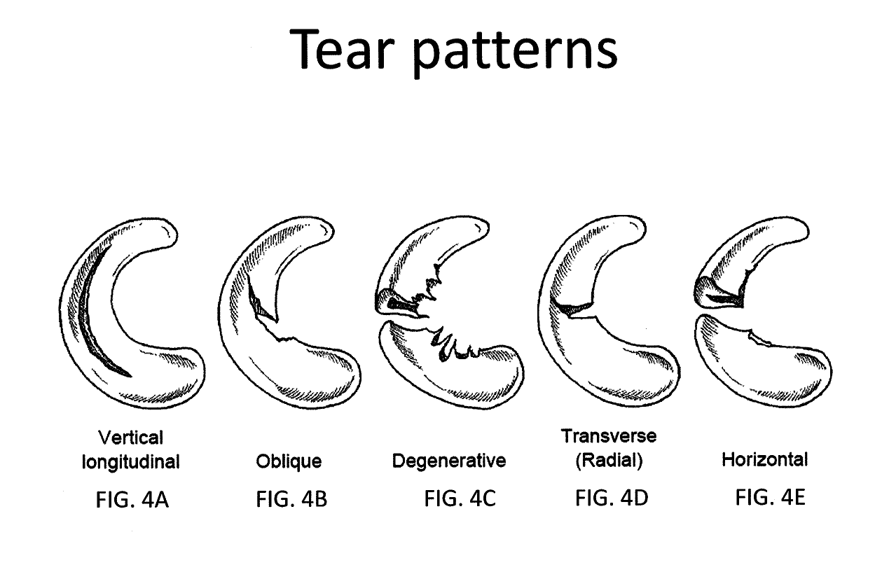

### DETAILED DESCRIPTION

Described herein are sutures passers. These suture passers may be used arthroscopically, and may be used to pass one or more length of suture. In general, the suture passers described herein include an elongate body and a first jaw member (e.g., first jaw) extending from the distal end of the elongate body, wherein the first jaw is bent or bendable relative to the distal to proximal axis of the elongate body. In some variations the first jaw is hinged near the distal end region of the elongate body. Some variations of the suture passers described herein include a second jaw member (e.g., second jaw) that is configured to slide axially (proximally and distally) relative to the elongate body and/or first jaw. The first and second jaws may be configured to form a distal-facing opening into which tissue may be held. The suture passers described herein may also include a flexible, bendable, or pre-bent tissue penetrator for passing a suture through the tissue. The suture passer may also include a handle at the proximal end with one or more controls for actuating the first and/or second jaws and the tissue penetrator.

In some variations the suture passers described herein include an axially slideable second jaw that is configured to penetrate tissue. This configuration may allow the device to pass the suture in an angled pathway through the tissue, including “L-shaped” pathways within the tissue.

In some variations, described herein are suture passer having very narrow second jaws; the tissue penetrator may exit the second jaw from the side of the second jaw and extend across a distal-facing opening to engage an opening in the opposite jaw (e.g., the first jaw), where a suture may be secured and/or released. For example, the suture passers described herein may have a second jaw having a maximum diameter (e.g., maximum height) along the length of the second jaw of less than about 0.11 inches, 0.10 inches, 0.09 inches, 0.08 inches, 0.07 inches, 0.06 inches, 0.05 inches, 0.04 inches, 0.03 inches, 0.2 inches, 0.01 inches, etc. The second jaw may be any appropriate width. For example, the width may be approximately 0.15 inches.

In some variations, described herein are suture passers that do not include a second (e.g., lower) jaw, but that are instead configured so that a tissue penetrator (e.g., needle, ribbon, etc.) extend from the distal end region of the elongate member to engage a side region of the first jaw to pass a suture through the tissue. An elongate member may be any elongate structure extending from the proximal to distal end region of the device (e.g., cannula, tube, cylinder, arm, shaft, etc.).

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

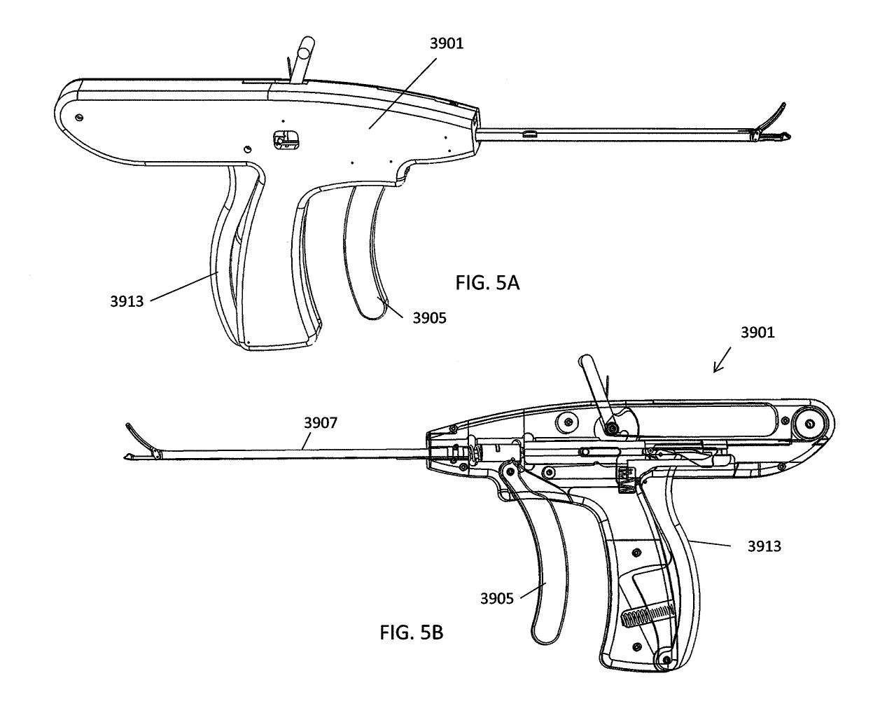

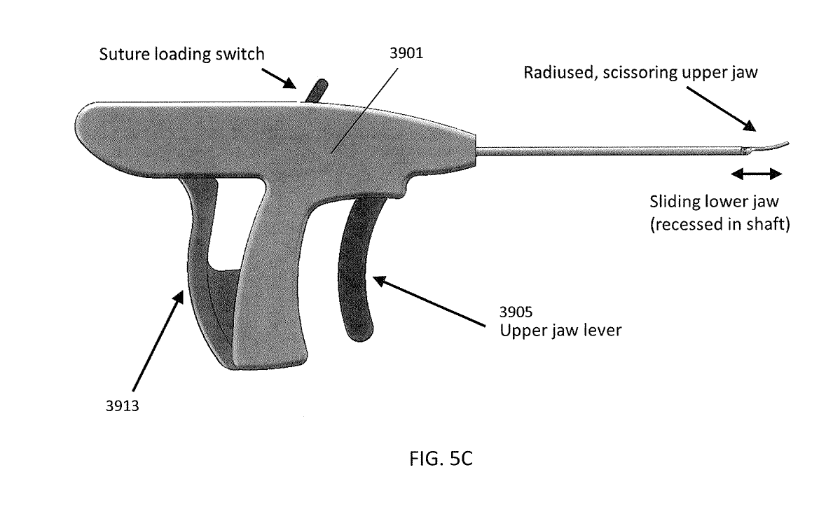

FIGS. 5A-5C illustrate suture passers that have a first jaw and a second jaw that may be controlled to form a distal-facing opening across which a tissue penetrator may extend to pass a suture through tissue between the first and second jaws. For example, in FIG. 5A, the suture passer of has a tissue penetrator that extends distally from a distal opening in the first jaw. In this example, the tissue penetrator travels in a sigmoidal (e.g., approximately “S-shaped”) path from the second to first jaw. In this variation, one or more length of a suture (including two lengths of the same suture, e.g., two ends of the same suture) can be loaded into the second jaw and passed from the second jaw, through the tissue and retained in the first jaw, to pass a length of suture through the tissue. The suture passer show in FIGS. 5A-5C is also configured so that the first (e.g., upper) jaw can pivot to assume a different angle relative to the elongate body of the device, and the second jaw is axially slideable or extendable distally from the distal end of the elongate member to form a distal-facing mouth with the first jaw. The proximal handle includes a plurality of controls for controlling the pivoting of the first jaw, the axial sliding of the second jaw, and the extension/retraction of the tissue penetrator from the second jaw.

FIG. 5B shows the device of FIG. 5A with the outer housing of the proximal handle 3901 removed, revealing some of the connections between the controls and the device. In FIG. 5B, the distal most control 3905, the proximal handle is configured as a trigger or lever that controls the motion of the first jaw (“first jaw control”). The first jaw control may be pulled to reduce the angle of the first jaw relative to the long axis of the elongate member 3907. In this variation the first jaw control is pinned and allowed to drive a tendon in the elongate member distally when compressed to drive the first jaw down (reducing the angle between the first jaw and the long axis of the elongate member). This pivoting motion may also be referred to as scissoring (scissoring motion).

A distal control 3913 is also configured as a lever or trigger, and may be squeezed or otherwise actuated to extend and/or retract the second jaw to form a distal-facing mouth with the first jaw, as shown in FIGS. 5A-5B. In some variations the control is further configured to control deployment of the tissue penetrator in the sigmoidal path. For example, in some variations squeezing the distal control after completely extending the second jaw may deploy the tissue penetrator from the second to the first jaw so that the distal end of the tissue penetrator extends out of the first jaw. As it extends between the first and second jaw, the tissue penetrator may carry a first length (bight) of suture through the tissue. Upon reaching the opposite jaw, the suture may be removed from the tissue penetrator and held (e.g., by a stripper) in the first jaw. Upon release of the distal control, the tissue penetrator may withdraw back into the second jaw. Actuating (e.g., squeezing) the distal control 3913 again may result in the extending the tissue penetrator (along with any second length of suture) back through the tissue from the second jaw to the first jaw, where the second length of suture can be retained. Alternately, in some variations, the controls (e.g., to control motion of the first and/or second jaw) may be separate from each other, and/or from extending/withdrawing the tissue penetrator. Additional controls may also be included in the proximal handle, include a suture loading control (e.g., switch, toggle, etc.) for loading and/or tensioning the suture within the second jaw.

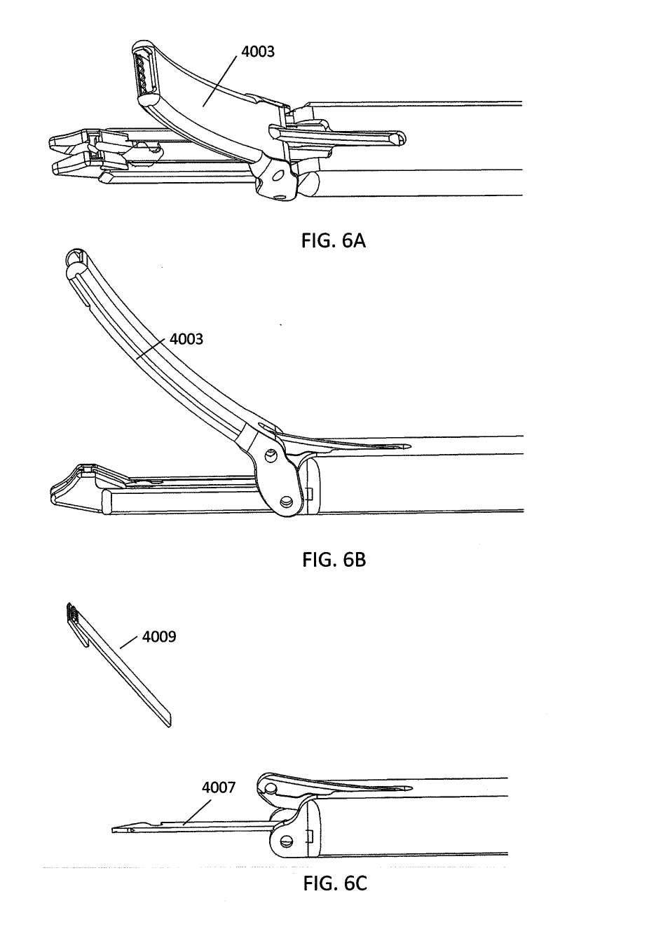

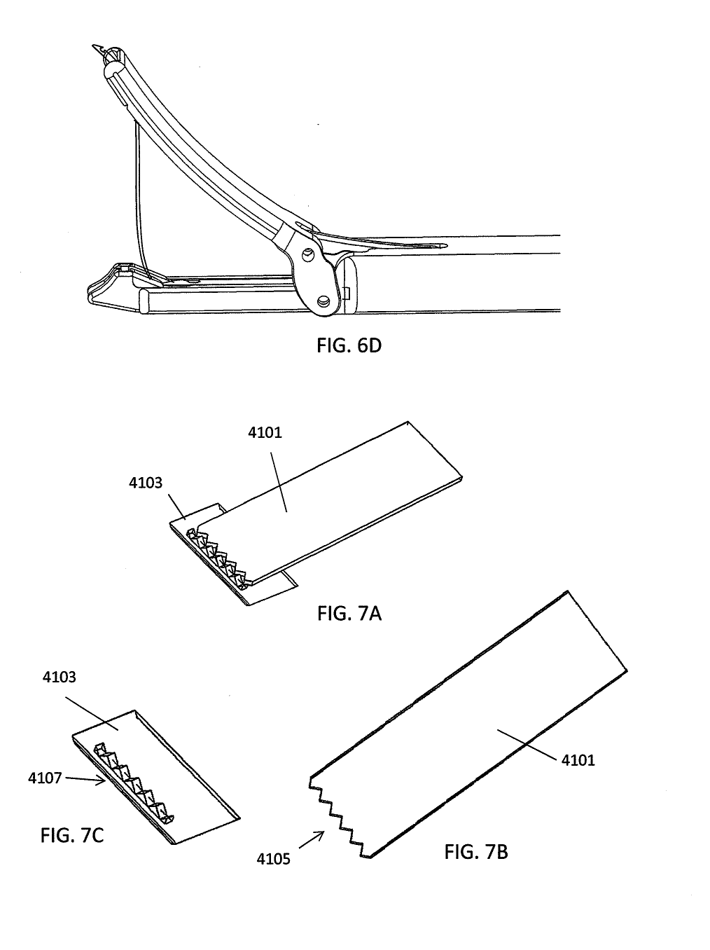

FIGS. 6A-6D show an enlarged view of the distal end of the device of FIGS. 5A-5C. For example, in FIGS. 6A and 6B the first jaw 4003 is thin and slightly radiused (e.g., curved), and is hinged to the elongate shaft region of the device. The first jaw is also connected to a control (handle, etc.) on the proximal handle by a push/pull member (tendon, wire, rod, etc.), allowing adjustment of the angle of the first jaw relative to the elongate member.

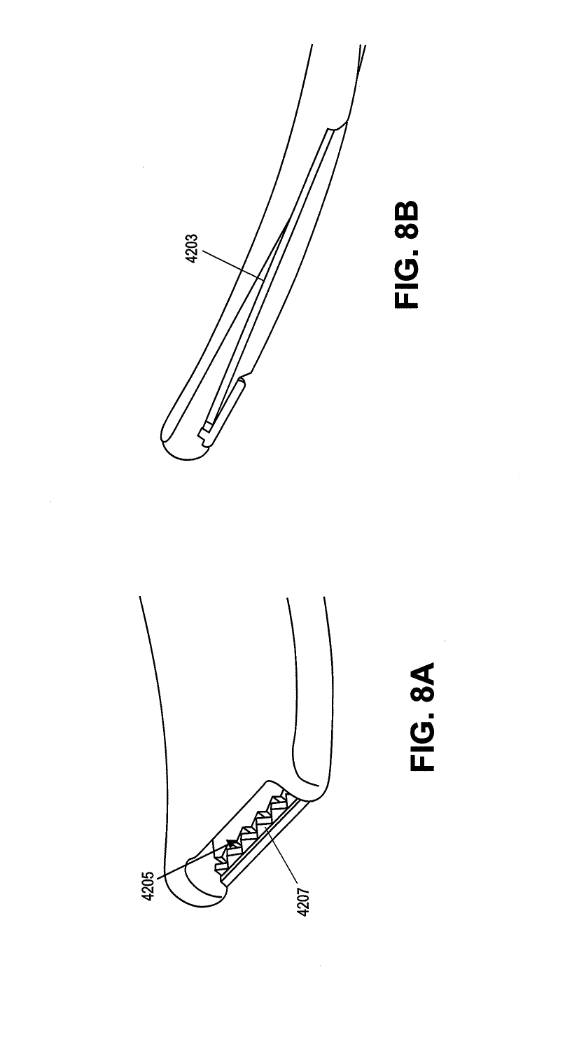

In FIG. 6C, the first and second jaws have been removed from the distal end of the device shown in FIG. 6B, revealing the tissue penetrator 4007 within the second jaw and a suture stripper 4009 (suture retainer) in the first jaw. FIG. 6D shows the distal end of the device of FIG. 6B after the tissue penetrator has been extended across the distal-facing mouth. FIGS. 8A and 8B illustrate one variation of a first jaw region having a suture stripper. In FIG. 8A, the suture stripper is visible from the distal opening at the distal end of the jaw. In this example, the stripper includes a stripper plate 4203 with a saw-toothed edge 4205. The jaw also includes a receiver region for the stripper plate having a sawtooth edge 4207.

FIGS. 7A-7C show greater detail on one variation of a suture stripper that may be used. This variation is the same as the variation shown in FIGS. 8A and 8B. Although the examples provided herein show the suture stripper in the first jaw, in some variations a suture stripper may be present on the second jaw (e.g., where the tissue penetrator is configured to pass a length of suture from the first jaw to the second jaw). In FIG. 7A, the stripper includes a flexible plate 4101 that is fixed at the proximal end (e.g. to the first jaw), and pressed against a receiving plate 4103 at the distal end 4105. In some variations the receiver is not a separate receiving plate, but merely a region of the jaw. Either or both the suture stripper plate 4101 and the receiver 4103 may include an edge that is adapted to catch the suture. In FIGS. 7A-7C, both the plate 4101 and receiver 4103 include edges having teeth 4105 and 4107. In this example the teeth are saw-tooth structures that are adjacent (or abutting) in the first jaw. The tissue penetrator may pass between the plate 4101 and the receiver 4103 by deflecting the plate 4101; as the end of the tissue penetrator passes the edges 4105 and 4107, a length of suture held by the tissue penetrator may be caught by the stripper and held between the plate and receiver as the tissue penetrator is withdrawn. In practice, a suture passer having a distally-extending tissue penetrator (including a pre-tied knot) may be used to repair a tissue such as the meniscus of the knee.

The devices and methods described herein may be used to pass a loop of suture and specifically, may be used to form a vertical or horizontal stitch to repair tissue. When repairing the meniscus, a vertical stitch typically provides the strongest repair with the least amount of displacement relative to horizontal stitches or other “all-inside” approaches. The devices and methods described herein may also be referred to as “all-inside” devices and meniscal repair techniques allow the meniscus to be sutured directly. The suture passers described herein may place a fully-circumferential, vertical stitch around meniscal tears. This stitch may provide uniform compression along the entire height of the meniscus and maintain coaptation of the tear at both the inferior and superior meniscal surfaces. Further, because of the jaw and needle configuration, the distal extending tissue penetrator does not penetrate the capsule wall, reducing or eliminating risk to posterior neurovascular structures. These features may allow a greater healing response due to complete tissue coaptation along the entire substance of the tear, improved clinical outcomes due to the greater healing response and to the anatomic reduction and fixation of the meniscus tear, may avoid scalloping or puckering of the meniscus, and may result in less extrusion or peripheralization of the meniscus caused by over-tensioning of suture or hybrid tensioners to the capsule. These devices can also be used to treat radial, horizontal, flap, and other complex tears in addition to longitudinal tears.

In some variations, the suture passer devices described herein can be fired blindly where arthroscopy camera access is poor, as knee structures are protected from the needle path.

Returning now to FIGS. 5A-5C, as mentioned above, the device (e.g., in FIG. 5C) has a scissoring first jaw that is curved (radiused). This curve may be configured to follow the radius of the femoral condyle. The second jaw in this example is relatively straight. The second jaw may be recessed (partially or completely) into the shaft, and may slide proximal-to-distal in order to slide under the meniscus along the tibial plateau after the first jaw is in place along the superior surface of the meniscus. The second jaw in this example contains a flexible needle, which moves vertically from the second to first jaw.

In some variations a knot of suture may be passed through tissue using a suture passer as describe above in which a pre-tide knot is used to help secure the length of suture being passed to the device. For example, in some variations an end region of one or both (in variations in which two lengths of suture are being passed) lengths of suture are knotted, and this pre-tied knot may be passed through the tissue by the tissue penetrator. The pre-tied knot may or may not include a leader snare. For example, in some variations two lengths of suture (from the same elongate suture) may be passed through a tissue; both lengths may be pre-knotted, however only one of the pre-tied knots may include a leader snare and be configured to allow another length of suture to be pulled through using the leader snare.

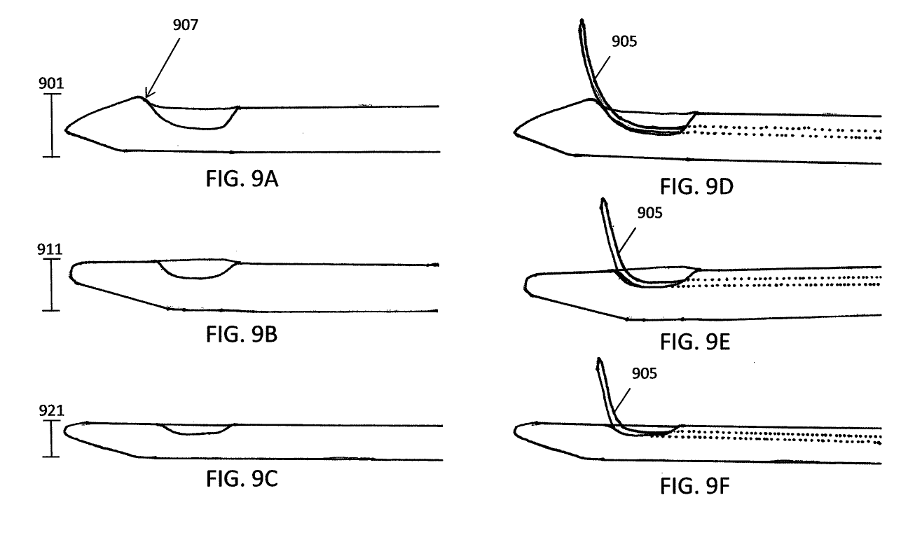

In some variations, the suture passers described herein may include a second (e.g., lower) jaw that is thin (e.g., <0.11 inches in diameter at the widest point). In general, thinner second jaws may be inserted into narrower and difficult to access body regions. In some variations, in which the second jaw houses the tissue penetrator and the tissue penetrator extends across the distal-facing opening formed between the first and second jaw, the second jaw may include a deflection ramp or deflection structure to help deflect the tissue penetrator out of the jaw and across the distal-facing opening. The deflection ram or deflection structure in some variations may form a widened region of the second jaw. Although it was initially believed that this enlarged deflection region was necessary to provide sufficient deflection and control of the motion of the tissue penetration, recent information suggest that this may not be necessary, particularly when using a pre-bent or pre-biased shape memory material to form the tissue penetrator. Thus, as shown in FIGS. 9A to 9C, second jaws housing a tissue penetrator may be used, wherein each one has a different thickness and/or different size deflection region. For example, in FIG. 9A the second jaw includes a deflection region 907 distal to the opening from which the tissue penetrator may be extended (extension of a tissue penetrator is illustrated in FIG. 9D). The widest diameter portion 901 of the jaw in this example is the deflection region 907. In some variations the widest diameter region is less than approximately 0.15 inches (e.g., less than about 0.14 inches, less than about 0.13 inches, less than about 0.12 inches, less than about 0.11 inches, less than about 0.10 inches).

Although a protruding deflection region may be helpful for steering the tissue penetrator/needle as it leaves the jaw, surprisingly, in some variations a protruding deflection member is not necessary, allowing the diameter of the jaw to be thinner. For example, in FIG. 9B, a jaw housing a tissue penetrator is shown without a protruding deflection member. FIG. 9E shows the jaw of FIG. 9B with a tissue penetrator 905 extending from the side of the jaw. In this example, the jaw is thinner than the example shown in FIG. 9A; the maximum diameter (e.g., maximum height) of the jaw 911 is less than about 0.10 inches (e.g., less than 0.09 inches, less than 0.08 inches, less than 0.07 inches, less than 0.06 inches, etc.). FIG. 9C shows another example in which the jaw is even thinner (e.g., less than 0.06 inches, less than 0.05 inches, less than 0.04 inches, less than 0.03 inches, etc.). In any of these examples the jaw may have a width. For example in some variations the width is between about 0.01 inches and about 0.15 inches. The tissue penetrator is typically thinner and narrower than the jaw so that it may fit within the jaw; the tissue penetrator (e.g., needle) may have a square, round, rectangular, or other cross-sectional area. In general, the tissue penetrator may be configured as a ribbon-shaped tissue penetrator, having a sharp (e.g., pointed, beveled, etc.) distal tip region, and a suture retaining region (e.g., hook, eyelet, etc.).

Any of the jaws illustrated in FIGS. 9A-9F may be used as a second or lower jaw for a suture passer as illustrated above (e.g., FIGS. 5A-6B). In general, the suture passers shown in FIGS. 5A-6B include first and second jaws having atraumatic (e.g., non-tissue penetrating) distal tip regions. Thus, as illustrated in these figures, the distal tip region of both jaws (first and second) are rounded and atruamatic so that they do not readily penetrate or cut the tissue. However, in some variations, the distal tip region of a jaw is tissue penetrating, allowing the jaw to be inserted into the tissue. In particular, it may be beneficial to have the axially slideable jaw (e.g., the second jaw) be tissue penetrating so that it can be extended into the tissue. This may allow the suture passer to pass a suture in an angle within the tissue (including at a right angle, e.g., to form an approximately “L” shape).

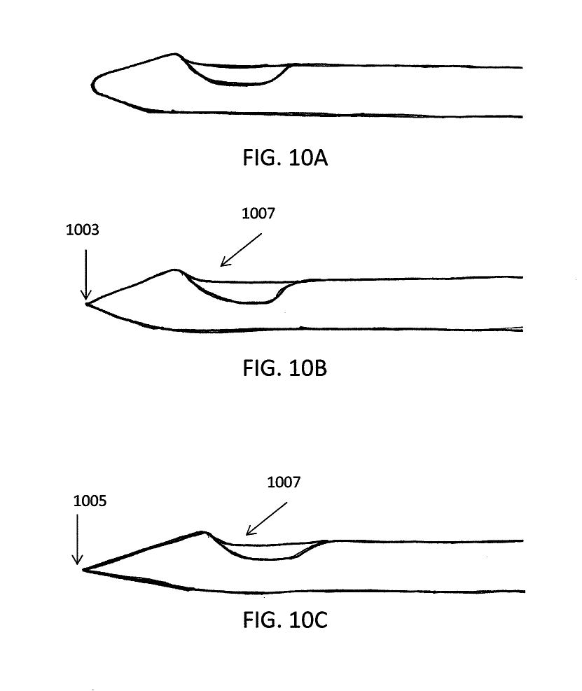

FIG. 10A shows an example of a rounded (e.g., atraumatic) distal tip region. FIGS. 10B and 10C illustrate variations of a jaw having a tissue penetrating distal tip region. A tissue penetrating distal tip region may be a sharp (e.g., pointed, beveled, serrated, etc.) distal region that is configured to cut into tissue when advanced distally. The tissue penetrating distal tip region may be configured to cut tissue by applying energy (e.g., RF energy, thermal energy, sonic/ultrasonic energy, etc.). In some variations the distal tip region is ablative. In some variations the distal tip region is a combination of sharp and energy applying. Although the distal tip region of FIG. 10A is shown as rounded, in some variations it may be configured as a tissue penetrating distal tip region by including the application of energy to the distal tip region. A tissue penetrating distal tip region may be adapted to penetrate any appropriate tissue, including soft tissues, cartilage, bone, etc. In some variations the tissue penetrating distal tip region is movable (e.g., rotating, vibrating, oscillating, etc.) in order to aid in cutting the tissue.

In FIG. 10B the distal tip region 1003 is pointed (sharp). This figure is shown as a side view; in some variations the distal tip region is configured to have a flat (e.g., having a thin, sharp edge), and thus has a width (not shown); in some variations the distal tip comes to a point. FIG. 10C shows another variation of a distal tip region of a jaw having a more tapered tissue penetrating distal tip region 1005. All of the jaws shown in FIGS. 10A-10C are configured to house a tissue penetrator (e.g., needle) and include a side exit region 1007 from which the suture-carrying tissue penetrator may extend or retract.

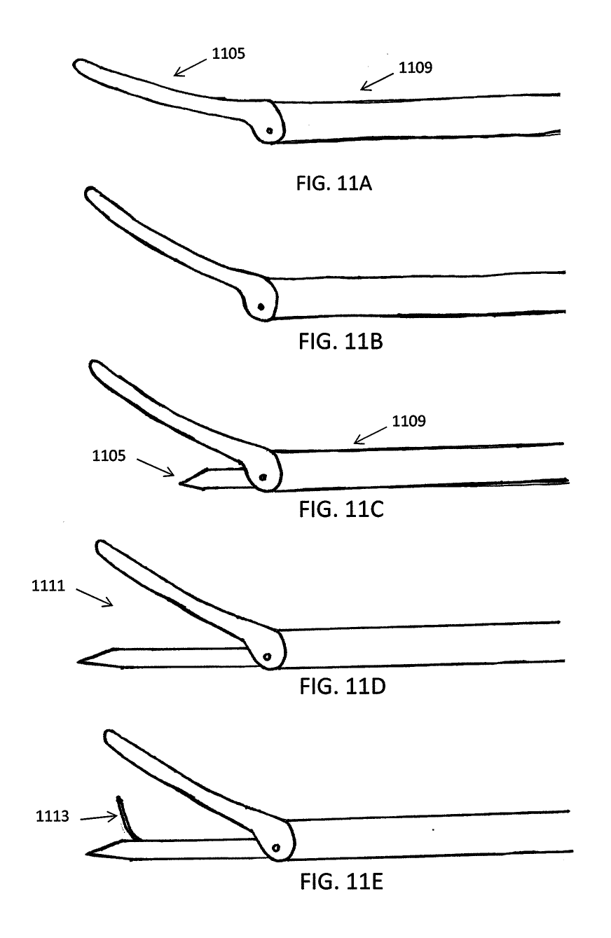

FIGS. 11A-11E illustrate operation of one variation of a suture passer including a tissue-penetrating distal tip region on a second (axially slideable) jaw. In this example, the first jaw 1105 is connected by a hinge point to the distal end region of an elongate member 1109. The elongate member may be connected proximally to a handle, as discussed above. In this example the first jaw may be bent relative to the long axis of the elongate member, as shown in FIG. 11B. In some variations the first jaw is fixed at an angle relative to the long axis of the elongate member 1109. In FIG. 11C, a tissue-penetrating distal tip of a second jaw 1105 extends distally from the distal end of the elongate member 1109. This variation of a second jaw is axially slideable relative to the elongate member, and is configured to extend/retract into the elongate member. As shown in FIGS. 11A and 11B, the second jaw may be configured to retract completely relative to the elongate member, so that it does not form a distal-facing opening with the first jaw.

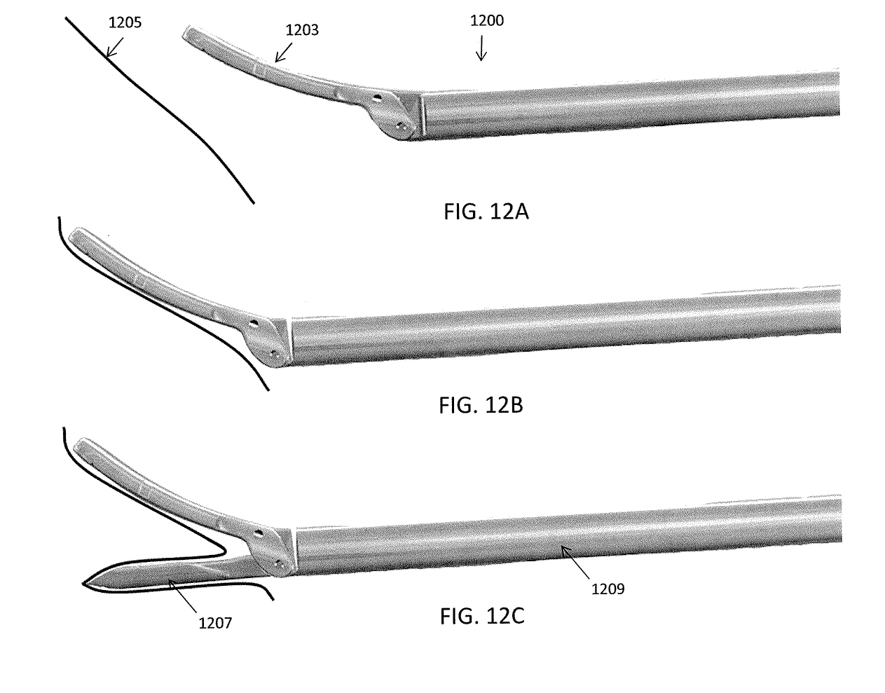

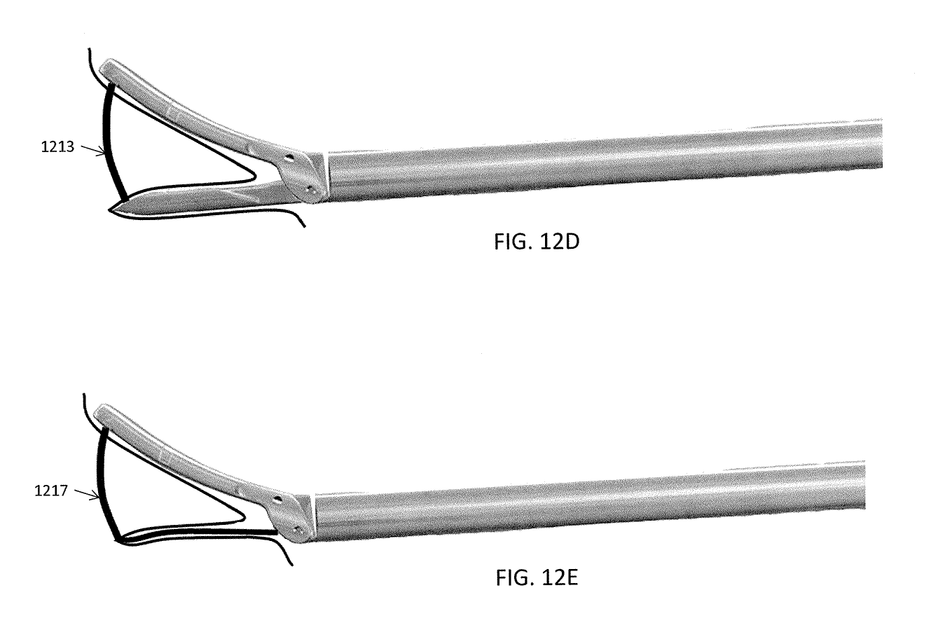

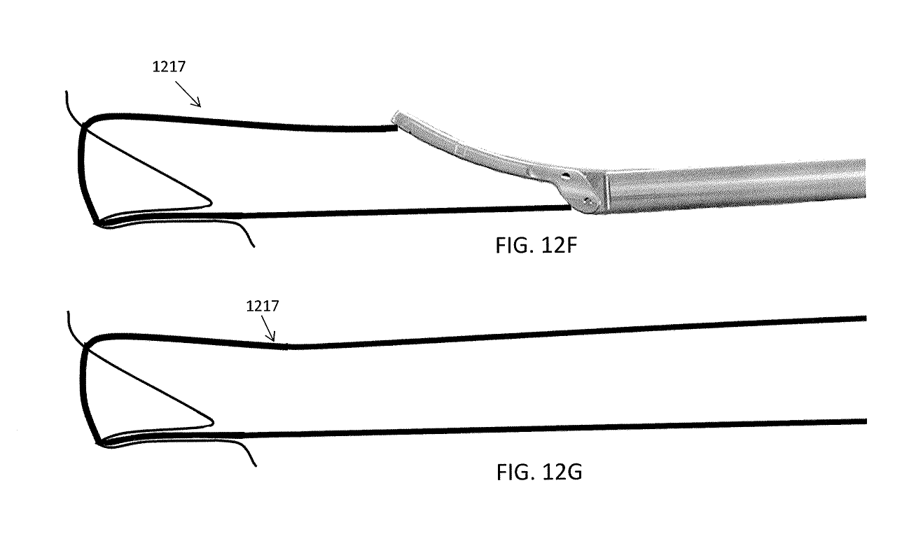

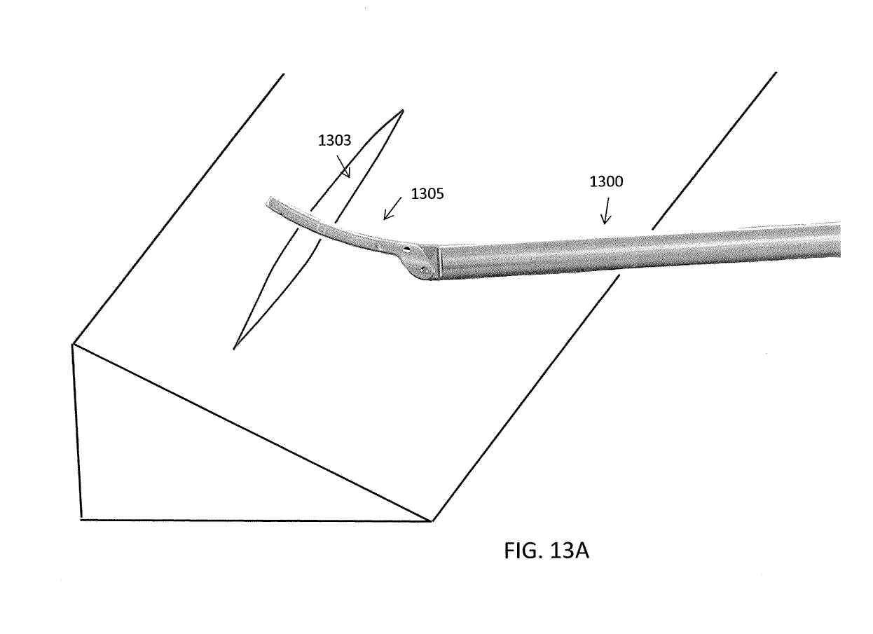

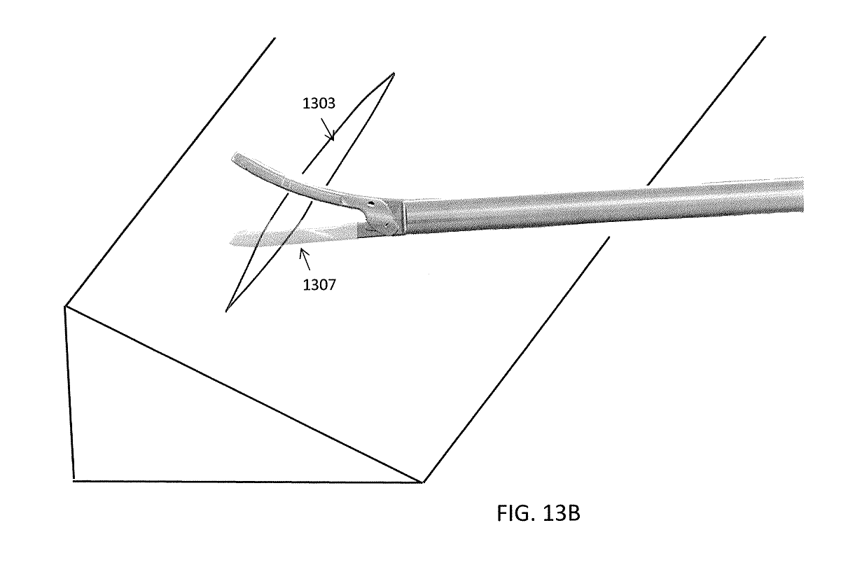

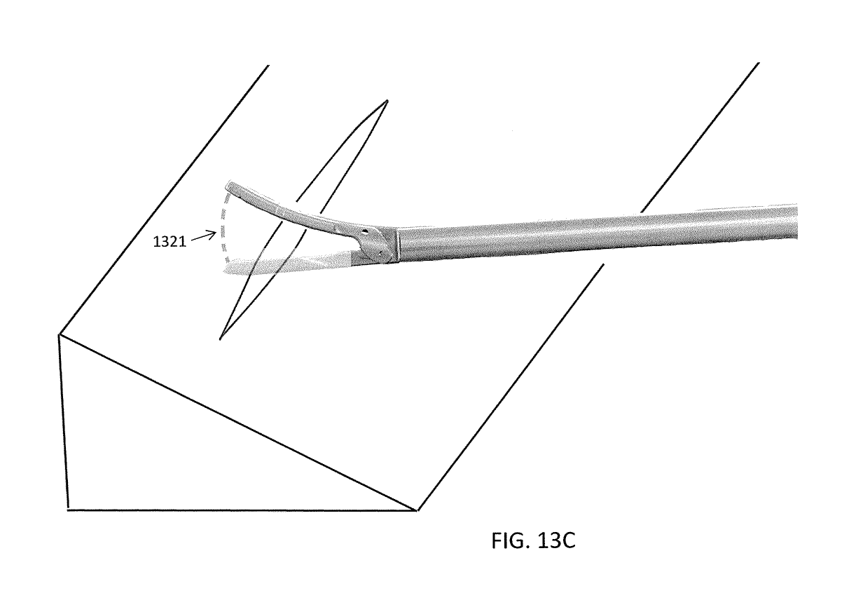

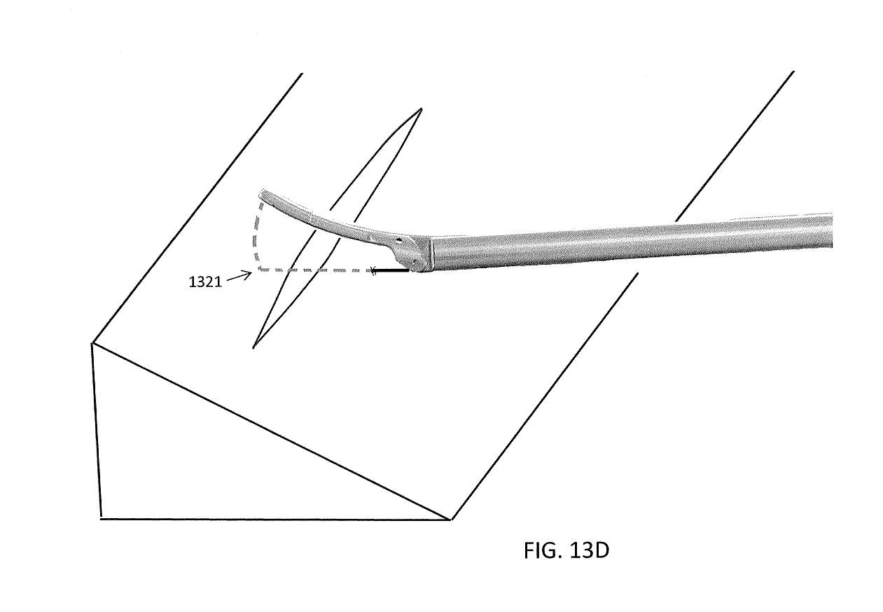

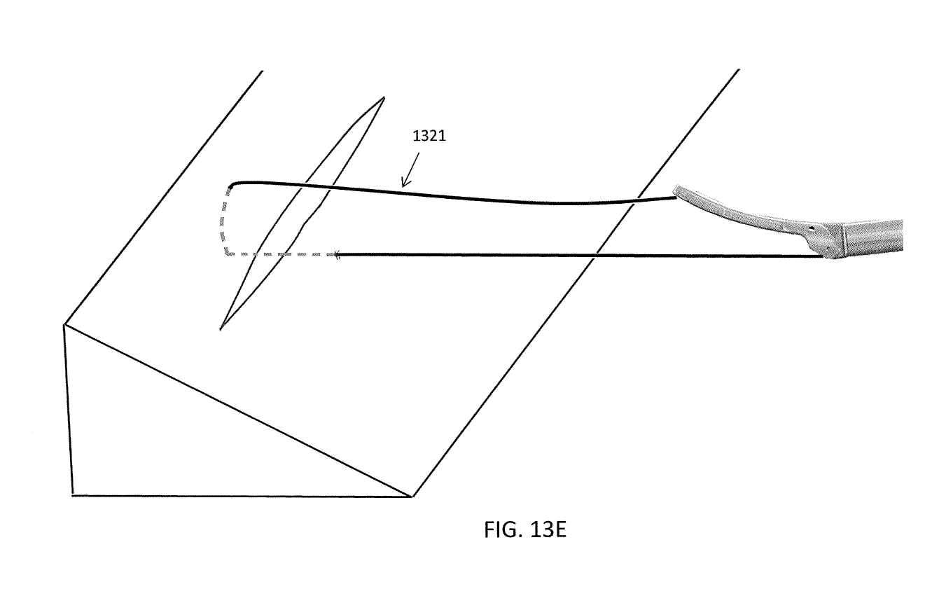

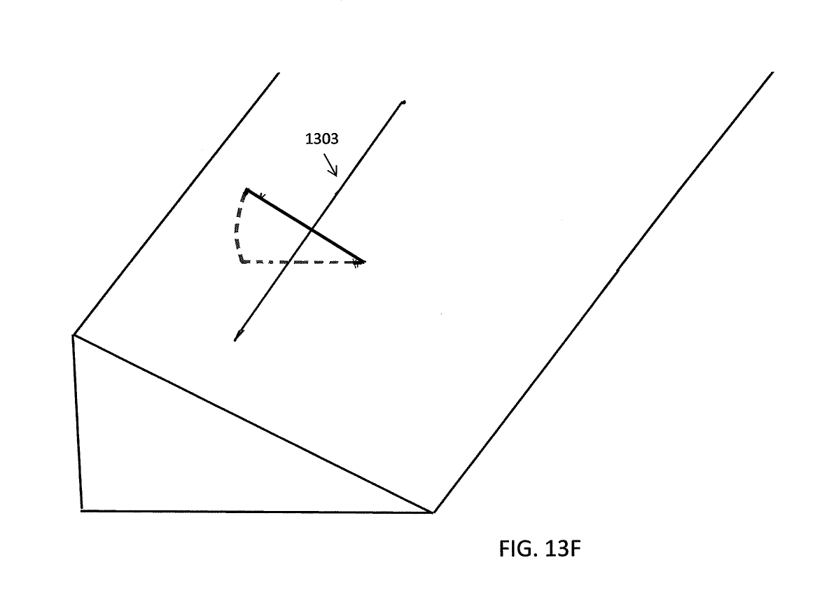

Sliding the second jaw distally may drive the tissue-penetrating distal tip of the second jaw into a tissue, as illustrated below (e.g. FIGS. 12C and 13B). Sliding the second jaw distally also forms a distal-facing mouth or opening 1111 which may hold tissue to be sutured. The second jaw may be configured to have one or more predetermined (e.g. “stop”) positions; the second jaw may be configured to extend to this predetermined position when the control is actuated, or the predetermined position(s) may be indicated by a friction point, click, or locking as the second jaw is advanced. In some variations, once the second jaw is extended, a suture passing tissue penetrator (e.g., needle element) 1113 may be extended between the first and second jaws, as illustrated in FIG. 11E. The tissue penetrator may be housed within either the first or second (shown in this example as housed within the second) jaw, and may be extended completely across the distal-facing opening to pass a suture between the first and second jaws. The suture may be pre-loaded on either the tissue penetrator or within one of the jaws. The suture may be either pushed (e.g., from the jaw housing the tissue penetrator) or pulled (from the jaw opposite the jaw housing the tissue penetrator) by the tissue penetrator. The suture, once passed between the jaws, may be held by the jaw to which it was passed. In variations in which the suture is passed by pushing the suture from the jaw housing the tissue penetrator, the suture may be retained by the opposite jaw and the tissue penetrator may be retracted back to the jaw from which it was extended.

Another example passing a suture using a suture passer having a jaw with a tissue-penetrating distal tip is shown in FIGS. 12A-12G. In this example, first arm 1203 of the suture passer 1200 is positioned adjacent the tissue to be sutured 1205, as illustrated in FIGS. 12A and 12B. This step (and the entire procedure) may be performed arthroscopically, though a small incision, tube, or portal. The tissue to be sutured may be any appropriate tissue, including meniscal (e.g., knee meniscus) tissue, shoulder (e.g., rotator cuff), back/spinal tissue (e.g., disc annulus, etc.). The tissue-penetrating distal end of the second jaw 1207 may then be slid distally to extend from the distal end of the elongate body 1209 of the suture passer. As the second jaw is extended distally, it cuts into the tissue 1205, so that the target tissue to be sutured is held between the first and second jaws. As shown in FIG. 12D, a tissue penetrator 1213 may then be extended between the first and second jaws, to pass a suture between the first and second jaws. Once the suture has been passed, the second jaw may be retracted, as illustrated in FIG. 12E. In this example, the suture 1217 has been passed through the tissue following both the tissue penetrator path and the path of the second jaw, resulting in an approximately L-shaped passage through the tissue, as shown. Thereafter, the suture passer may be removed from the tissue, as shown in FIGS. 12F and 12G, leaving the suture 1217 in place. The suture may then be tied off (e.g., knotted) or passed through another portion of the tissue (e.g., by re-loading into the suture passer).

FIGS. 13A-13F illustrate another variation of the method shown in FIG. 12A-12F, in which a tissue-penetrating distal tip region of a jaw is used to penetrate the tissue and pass a suture at an angel (e.g., in an approximately “L-shape”) within the tissue. As shown in FIG. 13A, the suture passer 1300 is positioned with the first jaw 1303 adjacent to the tissue. In this example the tissue may be a meniscus. The first jaw is positioned over a tear in the tissue 1303 so that a suture may be passed to repair the tear in the tissue. In FIG. 13B the second jaw 1307, which has a tissue penetrating distal tip region, is slide axially relative to the elongate body of the suture passer so that that the second jaw extend into the tissue, and through the tear in the tissue, in this example. In FIG. 13C the tissue penetrator is then extended between the first and second jaws, through the tissue between the distal-facing opening, to pass a suture 1321 between the jaws. Thereafter, as shown in FIG. 13D, the second jaw may be retracted by sliding proximally, leaving the suture 1321 in position following behind the retracted jaw. The suture passer may be removed from the tissue, as shown in FIG. 13E, leaving the suture passed through the tissue, still connected to the suture passer. The resulting loop of suture may then be tightened to draw the ends of the tear 1303 in the tissue together, and the suture may be secured (e.g., tied off), as illustrated in FIG. 13F. In some variations the suture may be knotted or tied using a pre-tied knot.

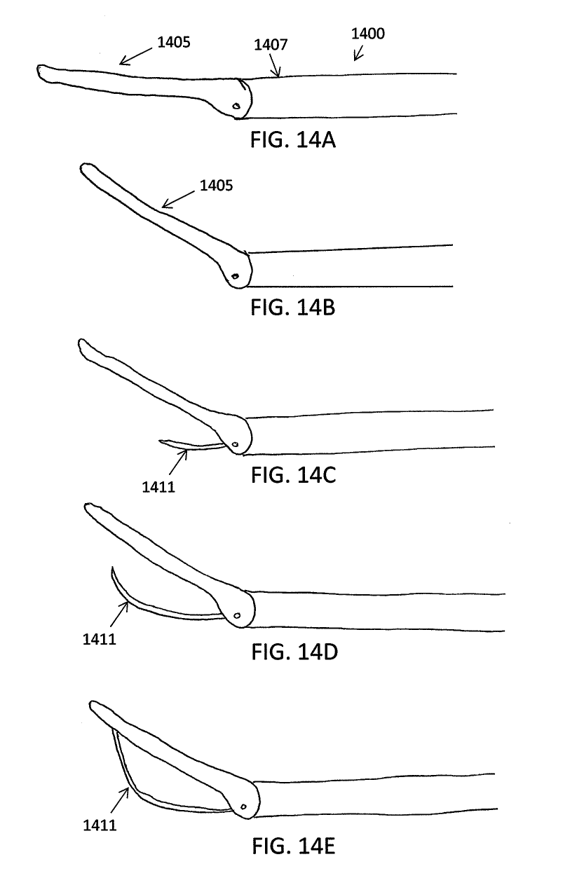

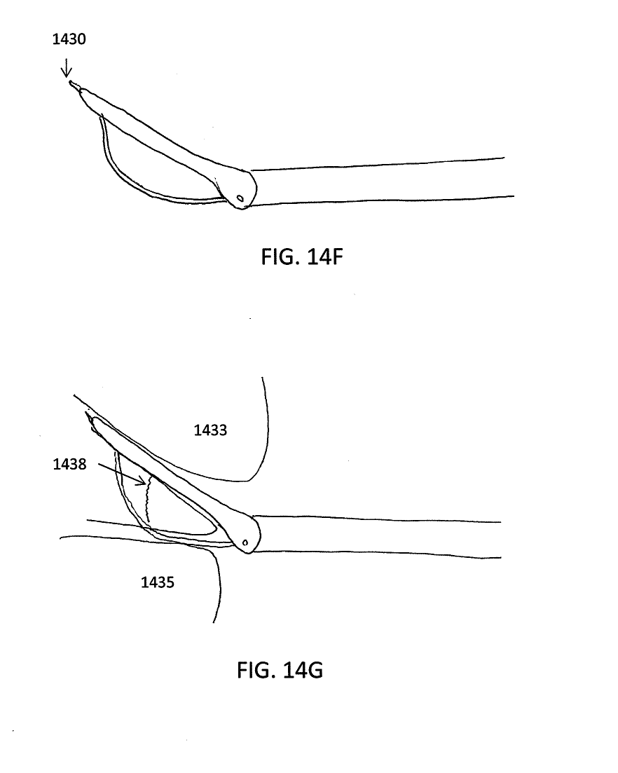

In some variations a suture passer may pass a suture substantially as illustrated and described above, but without the use of a second jaw. In such variations the tissue penetrator may be extended between the first jaw and the distal end region of the elongate body of the suture passer. FIGS. 14A-14G illustrate one variation of a suture passer that does not include a second (e.g., lower) jaw.

In FIG. 14A, the suture passer 1400 includes a bendable first jaw 1405 that is shown in this example as hinged to the distal end of the elongate body 1407 of the suture passer. In

FIG. 14B, the first jaw is shown bent relative to the elongate body member to form an approximately 30° angle with the long axis of the elongate body. In this example, the tissue penetrator of the suture passer is a shape memory material having a curved profile when not constrained by the suture passer. In FIG. 14A-G, the tissue penetrator is configured to extend from the distal end of the elongate body to a side region of the jaw 1405; in some variations the tissue penetrator is instead housed in the jaw to meet up with the suture passer at the distal end region of the elongate body 1407.

As shown in FIG. 14C, the tissue penetrator 1411 may extend from the elongate body and assume a curved shape as it is extended through the tissue (in FIG. 14D) to meet with the jaw as shown in FIG. 14E. In some variations the tissue penetrator is pre-loaded with the suture and pushes the suture through the tissue; alternatively, in some variations the suture is grabbed by the extended tissue penetrator and pulled through the tissue when retracting the tissue penetrator.

As shown in FIG. 14F, in some variations the jaw includes a deflection region and a distal opening from which the tissue penetrator may exit the jaw and be directed distally 1430. This variation of suture passers, which do not include a second jaw, may be used to suture any appropriate tissue, as mentioned above. For example, FIG. 14G shows the suture passer described above in FIGS. 14A-14F used to suture a torn meniscus. In this example, the jaw is positioned (as illustrated in FIG. 14A-14B) adjacent the superior surface of the meniscus, between the superior meniscal surface and the head of the femur 1433. The tissue penetrator may then be extended distally from the elongate body so that it passes between the inferior surface of the meniscus and the tibial plateau 1435 before passing into the meniscus on one side of a tear in the meniscus 1438. Thus, a suture may be passed between the superior and inferior surfaces of the meniscus; the suture passer may be removed (e.g., by retracting the tissue penetrator and withdrawing the jaw) and the suture left behind. The suture may then be re-loaded onto a suture passer (e.g., the same suture passer) and again passed through the meniscus to complete the loop of suture. The suture may be tied off, as mentioned above.

Although the description above is broken into parts and includes specific examples of variations of suture passers, any of the features or elements described in any particular example or section may be incorporated into any of the other embodiments. Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A suture passer device for passing a suture, the device comprising:
an elongate body extending distally and proximally along a long axis;
a first jaw extending from a distal end region of the elongate body wherein the first jaw is bent or bendable at an angle relative to the long axis;
a second jaw having a sharp, tissue penetrating distal tip, wherein the second jaw is configured to slide axially along the long axis distally and proximally relative to the elongate body, further wherein the first jaw and the second jaw form a distal-facing opening when the second jaw is extended distally and wherein the second jaw is retractable proximally so that it does not form the distal-facing opening with the first jaw; and
a tissue penetrator configured to extend across the distal-facing opening between the first jaw and the second jaw to pass a suture there between.

2. The device of claim 1, wherein the tissue penetrating distal tip of the second jaw comprises a sharp distal tip.

3. The device of claim 1, wherein the tissue penetrating distal tip of the second jaw comprises a sharp point at the distal tip.

4. The device of claim 1, wherein the first jaw comprises an atraumatic distal tip region.

5. The device of claim 1, wherein the first jaw is hinged to the distal end region of the elongate body.

6. The device of claim 1, wherein the first jaw is bent at a predetermined angle relative to the long axis of the elongate body.

7. The device of claim 1, wherein a second jaw is configured to retract into the elongate body.

8. The device of claim 1, further comprising a handle at a proximal end region of the elongate body.

9. The device of claim 1, wherein the tissue penetrator comprises a strip of shape-memory material.

10. The device of claim 1, wherein the tissue penetrator comprise a suture holding region.

11. The device of claim 1, wherein the tissue penetrator is housed in the second jaw and configured to extend from an opening on the side of the second jaw.

12. The device of claim 1, wherein the first jaw comprises a suture retainer configured to receive and hold a suture from the tissue penetrator.

13. A method of suturing tissue using a suture passer having an elongate body, a first jaw bent or bendable relative to the elongate body, and a sharp, tissue penetrating axially slideable second jaw, the method comprising:
positioning the first jaw of the suture passer adjacent a tissue with the second jaw retracted proximally relative to the elongate body;
axially sliding the second jaw distally to penetrate the tissue by extending the second jaw distally by relative to the elongate body;
extending a tissue penetrator through the tissue between the first and second jaws to pass a suture between the first and second jaws;
retracting the tissue penetrator from between the first and second jaws; and
sliding the second jaw proximally out of the tissue while leaving the suture in the tissue.

14. The method of claim 13, wherein positioning the first jaw comprises bending the first jaw relative to a long axis of the elongate body.

15. The method of claim 13, wherein positioning the first jaw comprises positioning the first jaw of the suture passer with a sharp tissue penetrating distal tip of the second jaw retracted into the elongate body.

16. The method of claim 13, wherein axially sliding the second jaw comprises extending a sharp, tissue penetrating distal tip region of the second jaw into the tissue.

17. The method of claim 13, wherein extending the tissue penetrator through the tissue comprises extending a tissue penetrator from a side of the second jaw to a side of the first jaw.

18. The method of claim 13, wherein extending the tissue penetrator comprises extending the tissue penetrator in a curve between the first and second jaws.

19. The method of claim 13, wherein extending the tissue penetrator comprises pushing a suture between the first and second jaws.

20. The method of claim 13, further comprising retaining the suture in the first jaw.

21. The method of claim 13, wherein retracting the tissue penetrator comprises retracting the tissue penetrator into the second jaw.

22. The method of claim 13, wherein the first jaw is positioned arthroscopically.
